(19) United States
(12) Patent Application Publication
(10) Pub. No.: US 2005/0113654 A1
(43) Pub. Date: May 26, 2005

(54) Body function monitoring mouth guard

(76) Inventors: Paul J. Weber, Fort Lauderdale, FL (US); Luiz B. Da Silva, Danville, CA (US); John P. Wooldridge, Kihei, HI (US)

Correspondence Address: John P. Wooldridge, 252 Kaipii Pl., Kihei, HI, 96753, US

(21) Appl. No.: 10/916,787

(22) Filed: Aug. 11, 2004

Related U.S. Application Data
(63) Continuation-in-part of application No. 09/939,979, filed on Aug. 27, 2001, now abandoned.

(51) Int. Cl. A61B 5/00

(52) U.S. Cl. 600/309; 600/549; 128/903

(57) ABSTRACT

A device is described that can be easily used to warn an athlete of potential risk of hyperthermia due to an elevated core body temperature. The mouth guard device continually monitors the users internal or core body temperature and emits an alarm signal when body temperature exceeds a preset value. This invention can protect the physical well being of an athlete as well as improve overall team performance.

This is a continuation-in-part of U.S. patent application Ser. No. 09/939,979, filed Aug. 27, 2001, titled “Temperature And Body Function Monitoring Mouth Guard” and incorporated herein by reference.

### BACKGROUND OF THE INVENTION

1. Field of the Invention

The present invention relates to protective athletic equipment, and more specifically, it relates to a device and method for monitoring internal or core temperature in an athlete to prevent the risk of hyperthermia.

2. Description of Related Art

Modern athletes at the turn of the second millennium are pushing the limits of their own endurance and their environmental limits even farther in hopes of better performance. It is estimated that two million people play organized football every year at the college, high school, or professional level, as well as, the junior and pee wee levels. Training for these sports can often occur on hot days leading to the possible risk of hyperthermia. Approximately four to six players die each year of hyperthermia.

The most important parameter to monitor on an athlete in order to prevent hyperthermia is the athlete's internal, or core temperature. Measuring the surface skin temperature with stickers, as is done in the anesthesiology setting in an operating room, is impractical and inaccurate. Placing a simple sticker on an athlete's forehead would be impractical and inaccurate because a helmet might cover the area and sweating may occur over the device. Just as in medicine, a true core temperature usually needs to be derived by testing a representative body cavity, such as the mouth, anus and rectum, or the ear canal. These areas are usually exposed to more direct internal body environments. Testing the armpit or nose may reveal inaccurate results, and is usually regarded as an accurate measure of core temperature in the medical community. Therefore, a need exists for a convenient device that can reduce the risk of hyperthermia and be easily used during sports.

There is a need for a device that monitors an athlete's internal or core temperature and can trigger an alarm when that temperature exceeds a preset value. The present invention fulfills this need, and further provides related advantages.

### SUMMARY OF THE INVENTION

An object of the present invention is to provide a device to monitor internal or core body temperature to reduce the risk of hyperthermia in athletes or others.

Another object is to provide a device for wirelessly transmitting information measured by a device that monitors internal or core body temperature to a receiver.

Another object of the present invention is to provide a mouth guard that will include means for determining a user (1) temperature, (2) blood sugar, blood oxygen (3) pulse rate (4) oxygen saturation level (5) blood acidity and (6) carbon dioxide level.

These and other objects will be apparent to those skilled in the art based on the disclosure herein.

The device is a temperature and body monitoring mouth guard worn by an athlete. It both protects the teeth and continuously monitors core body temperature. When the user's core body temperature exceeds a preset value (e.g., 104 F), an alarm is emitted to warn the athlete or others around him of a potential health hazard. This information can also be wirelessly transmitted to a computer for analysis, making it possible to monitor multiple athletes and improve the overall performance of the team.

An embodiment of the present invention is a mouth guard made of biocompatible material. The users upper and lower teeth fit within the mouth guard and the tongue protrudes in and around the mouth guard. A temperature sensor having minimal or no toxicity is contained in the mouth guard in a location having dose proximity to the oral mucosa, the inner side of the mouth or the tongue. This area is relatively representative of core temperature. A flap, bulge or protrusion may be added to bring the surface of the inner mouth guard into apposition separately with the tongue and provide more accurate temperature measurements. A protrusion in the front portion of the mouth guard is an ideal location for a light emitting diode to indicate a health warning. The lips of the user typically do not meet in this area when a large mouth guard is in place making it an easy and visible location to mount a warning mechanism.

The temperature sensor is placed close to the surface to insure quick temperature measurements of the local tissue. The temperature sensor can be placed in a variety of locations as long as it has contact with the tongue or the inner surface of the cheek. Electrical wires connect the temperature sensor to the control electronics. The electronic control circuit is powered by a battery which is turned on and off by the user with a switch to increase battery life. A light can be emitted to indicate that the device is activated. The on/off switch can also be heat activated to turn on when the temperature exceeds a preset value. When the athlete's core body temperature exceeds the preset value, the control circuit activates a warning mechanism to indicate that a dangerous body temperature has been reached. The warning mechanism could be a light emitting diode, a color changing LED, or a bright flashing LED. Alternative alarm indicators can also be used such as an LCD to display current temperature; a vibrating element can be used to signal the user to a potential hazard. The alarm could also emit an audible signal. The LED could also be replaced with a flavor release reservoir, which would be activated when a dangerous temperature is reached. It is important that the temperature response time is quick and accurate allowing the athlete to remove the mouth guard between plays without risking inaccurate measurements.

An RF transmitter can be incorporated into the mouth guard so that the measurements can be communicated to a central receiver, which would allow multiple athletes to be monitored at the same time. It is also possible to replace the temperature sensor with a sensor to monitor blood oxygen, respiratory flow and sound flutter indicative of snoring/sleeping problems. A more advanced embodiment of the present mouth guard utilizes multiple sensors that make the mouth guard useful not only for athletes but also patients while sleeping.

### BRIEF DESCRIPTION OF THE DIAGRAMS

The accompanying drawings, which are incorporated into and form part of this disclosure, illustrate embodiments of the invention and together with the descriptions, serve to explain the principles of the invention.

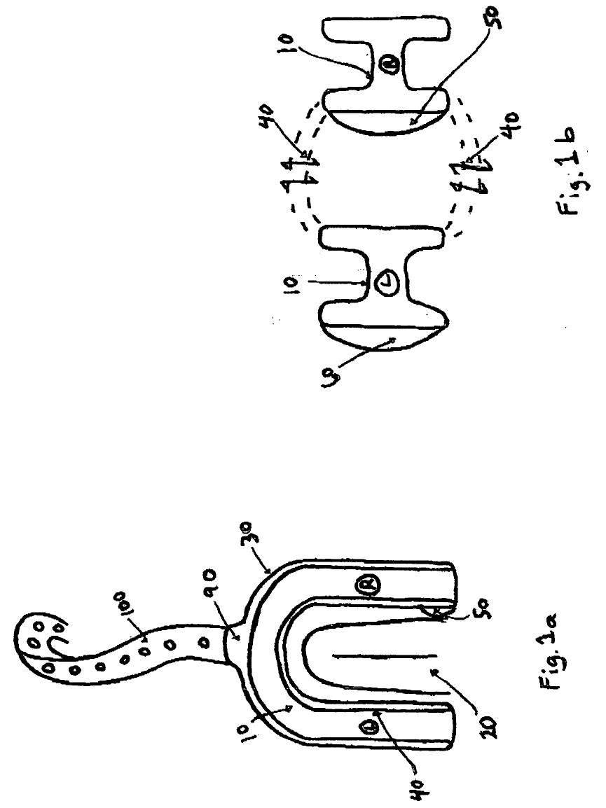

FIG. 1A is a top view of the mouth guard with the tongue in place.

FIG. 1B is a rear view of the mouth guard showing the shapes of the right and left side and the areas where the teeth insert

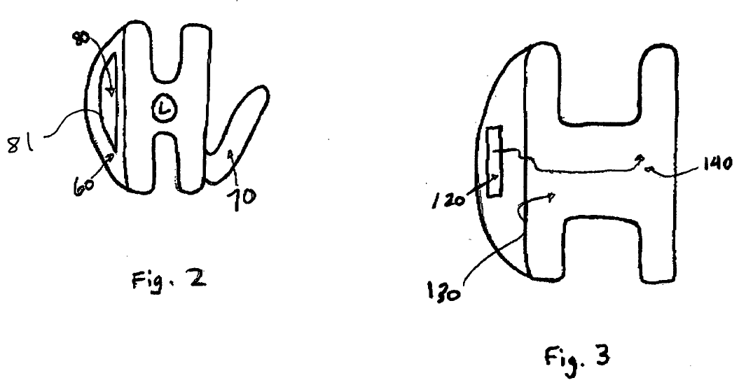

FIG. 2 shows alternate mechanisms for causing protrusion for an element of the mouth guard to come close to contacting either the side wall of the mouth where it is warm and where blood vessel flow is high, or the inner lateral surface of the tongue in which the blood vessel flow is even higher and more representative of a core temperature and core bodily functions.

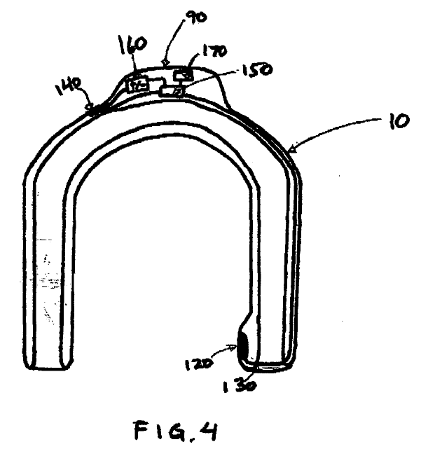

FIG. 3 shows a posterior view of the device and a sample bulge that is made allowing for the sensor to come into closer contact with either the inner surface of the mouth or the tongue, depending on which side is inside or out

FIG. 4 shows a top view and cutaway view of the mouth guard and the location of the battery microprocessor and stimulus-emitting device.

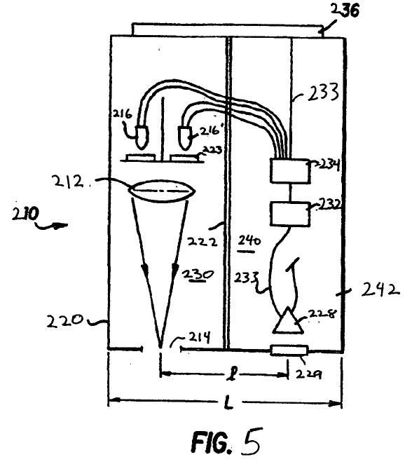

FIG. 5 shows a set-up of components usable in the for taking absorption, reflection or scattering data to determine the level of blood sugar, blood oxygen and oxygen saturation level, blood acidity and/or carbon dioxide level.

FIG. 6 shows a mouth guard with an embedded electromagnetic transmitter and detector pair.

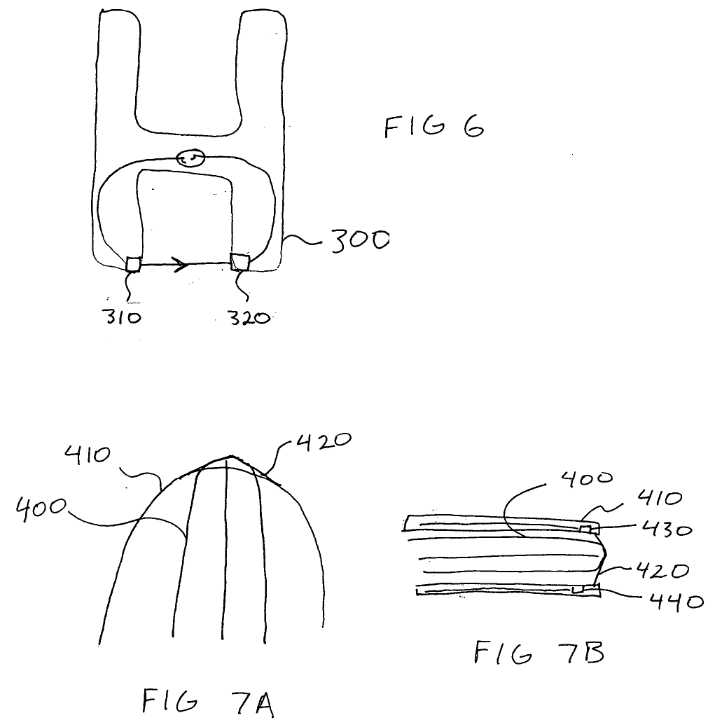

FIG. 7A shows a top view of an embodiment of the invention configured for absorption spectroscopy.

FIG. 7B shows a side view of the embodiment of FIG. 7A.

### DETAILED DESCRIPTION OF THE INVENTION

The invention is a device and method that monitors body functions such as the internal or core body temperature of an athlete to reduce the risk of hyperthermia. The device is a mouth guard worn by the athlete with at least one integrated sensor. In one embodiment, when core body temperature exceeds a preset value, an alarm sounds alerting the athlete and those around him that medical attention is necessary. An alternative embodiment of the mouth guard could contain multiple sensors to measure other body functions and wirelessly transmit this information to a computer for analysis. This information could be used to protect the individual athlete's physical health as well as improve the overall performance of the team. The device and method are not limited to the use by athletes. Embodiments could find use in other applications such as surgery or inherently dangerous activities such as underwater welding. The description of use by an athlete is exemplary.

FIG. 1A shows the key elements of an embodiment of the present mouth guard 10. The human tongue 20 can protrude in and around the mouth guard, which includes an outer peripheral rim 30, which lies exterior to the teeth when the teeth are in the biting position. The inner rim 40 of the mouth guard are near the teeth when the teeth are in the biting position, and it is this inner rim which comes into close proximity to the human tongue. The outer rim 30 comes in close proximity in its rear area to the oral mucosa, which is the inner side of the mouth, which can also be relatively representative of core temperature. A forced bulge or flap 50 is made in the inner lining 40, so that good apposition in measuring of the tongue may occur. The frontal extrusion area or a thickening area 90 of the peripheral rim 30 in which the retaining strap 100 is usually placed, is frequently cut off by athletes. Nonetheless, the protrusion 90 is a relatively comfortable area to mount excess material in the rim 30 because the lips typically do not meet in this area when a large mouth guard is put in place, and no rubbing or irritation occurs. Additionally, this area is usually very visible through the front lips on almost all athletes. This would be the ideal place to put a light-emitting diode, such as a red flashing diode to indicate a warning to another person.

FIG. 1B represents a posterior view of the mouth guard, both the right and left sides, showing the area 10 where the inner depression of the mouth guard is present, where the upper and lower teeth rest in the mouth guard. Inner rim 40 is drawn in a broken fashion to save space. Inner bulge 50 is shown on the posterior aspect of the mouth guard, which helps bring it into close contact with the tongue. Outer bulge 60 is placed in the posterior lateral aspect of the mouth guard in order to bring the mouth guard into close proximity for sensing temperature or other variables of the oral mucosa posteriorly, where they are less affected by breathing or other changes.

FIG. 2 represents an enlarged view of FIG. 1B, showing one segment only, the left posterior segment This view shows alternative ways to bring the bulge into greater apposition with the oral mucosa or tongue. The bulge 60, which would approximate the oral mucosa in the posterior view, could be enhanced by either a small inflation or insert 80 into a pocket 81. Additionally, a flap, bulge, protrusion, or any other geometric structure 70 may protrude off, especially the inferior portion of the posterior surface of the inner mouth guard, in order to bring it into apposition separately with the tongue.

FIG. 3 shows a cross sectional view through the posterior portion of the mouth guard showing one possible location for a temperature sensor 120. The temperature sensor 120 is placed close to the surface to insure that the sensor quickly responds to the local tissue temperature that contacts the mouth guard. The temperature sensor could be a thermistor (e.g., KC003T-ND from Keystone Thermometrics Corp, 967 Windfall Road, St Marys Pa.), a thermocouple, or integrated circuit temperature sensor (e.g., LM20, LM34, PC87365 from National semiconductor Corp. 1111 West Bardin Rd, Arlington Tex. 76017) Electrical wires 130 connect the temperature sensor to the control electronics. It is important that the temperature sensor and packaging within the device allow the system to have a temperature response time of less than 10 seconds. This would allow a football player to remove the mouth guard between downs without risking inaccurate measurements.

FIG. 4 shows one embodiment of the present invention. The temperature sensor 120 is located near a wall at the back end of the mouth guard where it ensures good contact with the tongue. In an alternative embodiment the sensor 120 could be placed on the outside surface to contact the inner surface of the cheek. Electrical wires 130 connect the temperature sensor element to the electronic control circuit 150. The electronic control circuit 150 is powered by a battery 160 and turned on by switch 140. The switch 140 is activated by the user before use and deactivated after use. When activated, the switch is recessed to minimize any irritation to the user. In one embodiment when the device is activated the light emitting diode (LED) 170 emits light to alert the user that the device is on and working. In another embodiment the switch 140 is a temperature-activated switch that opens when the temperature exceeds a preset temperature. A switch is desirable to increase battery life. The control circuit 150 includes a comparator to detect when the temperature measured by the sensor exceeds a preset value. When the temperature exceeds the preset value the control circuit 150 activates the light emitting diode 170 to indicate a dangerous temperature has been reached. In one embodiment the color of the LED 170 would change from green to red when the body temperature exceeds the preset value. In order to minimize power and extend battery life the control circuit 150 can flash the LED on and off with a low duty cycle (e.g., 10%).

In one embodiment the LED 170 continues to flash red even if the temperature returns below the preset value. This allows the user to determine if his temperature has been at a dangerous level. Coach's and other team players will be able to see the bright flashing red LED even when the mouth guard is within the mouth. The device can be reset by turning it off and on using switch 140. It is important to note that all the temperature sensor elements shown in FIG. 4 are encapsulated within the biocompatible mouth guard material. This protects the elements and user from direct contact In order to reduce risk, it is important that the materials used in the sensor elements have minimal or no toxicity. Although FIG. 4 shows that most of the device elements are located within the front protrusion 90, the device elements can be placed any where within the mouth guard.

An alternative embodiment replaces the LED 170 with at least one alarm indicator. These could include an LCD or organic light emitting diode display that can be used to display the current temperature. The alarm indicator could be a vibrating element that vibrates to signal the user of potential risk. The alarm could also be an audible alarm that sounds when a hazardous condition exists. Yet another alternative is to replace the LED with a flavor release reservoir in which sweet tastes may be stored or aromatics, such as cinnamon and aldehydes, or irritants such as capsaicin. These may be excreted into the saliva in order to alert the athlete that a hazard exists.

In another embodiment, an RF transmitter is incorporated into the mouth guard and the sensor measurements are communicated to a central receiver that can be used to monitor multiple players simultaneously. Another alternative is to replace the temperature sensor with a different sensor element For example, a saturated blood oxygen sensor similar to those used in pulsed oximetery could be used to monitor blood oxygen and heart rate. A more advanced mouth guard could incorporate multiple sensors (e.g., temperature, blood oxygen, heart rate, sodium and potassium level) and be used not only for sports but also for monitoring patients while sleeping. The wireless communication module could replace LED 170 and use simple RF communication or technology now available such as the Blue Tooth chip set.

The following is a suggestion of how the device could work. Pulse, temperature, sugar, oxygen and/or acidity are measured using the device during practice, and at various points during a game. A baseline during a game is plotted for each player. Standard deviations are derived from each value for each player. When the values exceed a preset limit such as one or two standard deviations then an alarm is transmitted to alert the trainer. The algorithm can recommend various treatments. All of the players can be graphed individually in small graphs with each value being a different color and standard deviation bands being different lighter-shaded bands surrounding the values, which are plotted against time of game or event The preset limits can be in fractions of standard deviations such as 1.34 standard deviations for an alarm etc. The device can be used to optimize team performance by pulling off and treating appropriately the players that are not optimal and substituting them with other players. Patterns of optimal health can be measured and used by the trainer in a predictive manner. Patterns of optimal behavior may be selected from these graphs.

An embodiment of the invention non-invasively measures the level of blood glucose by locating an optical transmitter and a detector on the surface of the oral mucosa. The electrical components of the device are battery powered. The battery can be located in a convenient location depending on the application, i.e., in the helmet of a football player. One embodiment transmits light into, e.g., the gum, the tongue or the inner wall of the mouth, where there is a relatively uniform profusion of blood, where the light will interact with the blood and be back-scattered onto the detector. Alternatively, the invention uses the principal of light transmission through a portion of the body that has relatively uniform profusion of blood in order to non-invasively measure blood glucose.

According to one embodiment of the invention utilizing near-IR interactance analysis techniques, near-IR light energy at bandwidths centering on one or more wavelengths of interest is passed through the skin and into the blood of a subject (The particular wavelengths and absorption cross-sections for blood constituents such as sugar, oxygen, acidity and carbon dioxide level are well known and readily ascertainable by those skilled in the art) A portion of the energy re-emerges from the blood of the test subject and is detected by a detector. Following wireless transmission and amplification of the detector-generated signal, the amplified output is processed into an output signal indicating the amount of the blood constituent under test, e.g., glucose, in the subject's blood. The output signal drives a display device for providing a visual display of blood glucose content.

According to another embodiment of the invention utilizing near-IR transmission analysis techniques, near-IR light energy at bandwidths centering on one or more wavelengths of interest is transmitted through, e.g., the gum, the tongue or the inner wall of the mouth, of a test subject The near-IR energy emerges from the test subject, generally opposite the near-IR source, and is detected by a detector. Following amplification of the detector-generated signal, the amplified output is processed into an output signal indicating the amount of glucose in the subject's blood.

In one embodiment utilizing near-IR interactance, the instrument, including near-infrared source, transmitter, detector, in some cases, an amplifier, and wireless transmitter are contained in an athletic-type mouth guard. Data processing circuitry and readouts are remotely located. Infrared emitting diodes (IREDs) disposed in one chamber of the unit are focused to transmit near-IR energy of preselected wavelength(s) to, e.g., the gum, the tongue or the inner wall of the mouth. The near-IR energy interacts with the constituents of the blood and is re-emitted or scattered onto the detector. A detector housed within a second chamber of the unit is disposed a distance from the emitter and collects this energy. The detected signal is amplified and data processed into a signal indicative of the amount of glucose in the blood. This signal is then wirelessly transmitted to a readout device (preferably a digital readout) for recordation and analysis.

In the interactance blood glucose embodiment 210 illustrated in FIG. 5, included is one or more means for providing at least one battery powered point source of near-infrared energy of a predetermined bandwidth of interest which is positioned within a first chamber 230 of the instrument 210. The near-infrared point source means is positioned so that near-infrared energy being emitted from the point source means will be focused by lens 212 through window 214 and onto the mouth tissue of the test subject The near-infrared point source means may comprise one or a plurality of battery-powered infrared emitting diodes (IREDs). Two such IREDs 216 and 216 are visible in the embodiment illustrated in FIG. 5. In other embodiments employing a plurality of IREDs, three, four or more IREDs may be utilized as the point source means.

Narrow bandpass optical filters can be provided between the infrared emitting diodes and the lens 212. According to this embodiment, a filter 223 is positioned between each IRED and lens 212 for filtering near infrared radiation exiting each IRED and thereby allowing a narrow band of near-infrared radiation of predetermined wavelength to pass through the filter and lens 212. Utilization of narrow bandpass optical filters provides for specific wavelength selection independent of the center wavelengths of the particular infrared emitting diodes being used. Measurements can be taken inside the half power bandwidth of the IREDs, or alternatively, outside the half power bandwidth of the IREDs.

A battery powered optical detector, illustrated schematically FIG. 5 and designated by reference numeral 228, is disposed within a lower end portion 242 of a second chamber 240. Inner wall 992 is positioned between detector 228 and lens 212, thereby providing an optically isolating mask, which prevents near infrared radiation from the point source means and/or lens 212 from impinging directly on detector 228. A near-infrared optical detector 228 generates an electrical signal when near-infrared radiation is detected thereby.

The optical detector 228 is connected to the input of an electrical signal amplifier 232 by suitable electrical conducting means 233. Amplifier 232 may be an inexpensive integrated circuit (IC) signal amplifier, and amplifies the signals generated when near-IR energy strikes detector 228. The output of amplifier 232 is fed to a wireless transmitter 236, which provides a signal to a remotely located data processor and readout device. Internal control logic and battery module 234 is provided for controlling and powering the IREDs, the detector, the amplifier and the wireless transmitter The readout device 236 may have a digital display for directly displaying the amount of glucose present in the subject's blood.

The embodiment of FIG. 5 includes an optical filter 229 for shielding all but the desired near-IR energy from detector 228. Filter 229 and window 214 are positioned for direct contact with the membrane or mouth tissue of the test subject. An optically clear window can be employed in lieu of filter 229, if desired.

The chamber 230 and its internal components may be physically separated from the chamber 240 and its components. In such a case, it may me desirable to include a separate, self-contained control and battery power supply within unit 230, thereby avoiding the need for wires to be routed from chamber 240 to chamber 230. By separating these two chambers, each can be placed within the mouth guard in any position where it would be desirable to have electromagnetic radiation traverse or be reflected or scattered from. Examples of such embodiments follow. It should be noted that the configuration of the chambers 230 and 240 is exemplary only, and that the main point to be taken from this description is that an electromagnetic source can be embedded within the mouth guard in such a position that its output will traverse through, or be reflected or scattered from, mouth tissue which has an adequate profusion of blood, whereby the electromagnetic radiation will interact with the blood constituent and then be collected by at least one detector that has been embedded in a mouthpiece. The unit 210 may be appropriately shaped to fit within the cavity of pocket 81 of FIG. 2. It may be desirable to omit certain elements from either chamber, e.g., the filters may be unnecessary, as well as the lens and optical window. Any variation is within the scope of this invention.

A variety of embodiments are capable of carrying out the transmittance analysis. One set-up embeds a transmitter 310 and a detector 320 in the mouthpiece 300 in such a way that electromagnetic radiation can pass through the gums to measure blood constituents. In one configuration, the transmitter and detector are located in the mouthpiece so that they align with each other between adjacent teeth, to avoid being blocked by a tooth. Another set-up embeds a transmitter in either the upper or lower mouth guard as illustrated in FIGS. 7A and 7B. The detector is located in the other mouth guard directly across from the transmitter so that light can transmit directly through the tongue. FIG. 7A shows a top view of the position of the tongue 400 as it aligns under the top mouth guard 410 and against the stop 420. FIG. 7B shows a side view of FIG. 7A. FIG. 7B shows an optical transmitter 430, such as an IRED, located in the top mouth guard 410 and aligned with a detector 440 such that electromagnetic radiation will pass through the tongue to enable detection of blood constituents under test Blood glucose measurements have been described in U.S. Pat. Nos. 6,151,517; 5,028,787; 5,077,476; 5,216,598; 5,582,169 and 5,706,821, all incorporated herein by reference.

Pulse oximetry is a non-invasive medical technique useful for measuring certain vascular conditions. In practice of the technique, light is passed through a portion of a patient's body that contains arterial blood flow. An optical sensor is used to detect the light that has passed through the body, and variations in the detected light at various wavelengths are then used to determine arterial oxygen saturation and/or pulse rates. Oxygen saturation may be calculated using some form of the classical absorption equation known as Beer's Law. The embodiments of FIGS. 5-7B are adaptable for pulse oximetry. Pulse oximetry is described in U.S. Pat. Nos. 6,421,549; RE28990; 4,086,915; 4,407,290; 4,807,631; 5,111,817; 5,411,024; 5,431,159; 5,448,991; 5,725,480; 5,820,550; 6,064,898; 6,151,107 and 6,226,540, all incorporated herein by reference.

The concentration (and thus the partial pressure) of certain gases, such as carbon dioxide and oxygen and the pH (hydrogen ion concentration) in arterial blood are determined without the need for contacting the blood by the absorption of electromagnetic radiation of particular wavelengths resulting from the constituent of interest is noted as a measurement from which the concentration of that constituent in arterial blood is calculated. The embodiments of FIGS. 5-7B are adaptable for measurement of blood acidity and carbon dioxide. See U.S. Pat. Nos. 4,041,932; 3,659,586; 3,795,239; 3,810,460; 3,825,342 and 3,848,580, all incorporated herein by reference.

The above descriptions and illustrations are only by way of example and are not to be taken as limiting the invention in any manner. One skilled in the art can substitute known equivalents for the structures and means described. The full scope and definition of the invention, therefore, is set forth in the following claims.

1. A body function monitor; comprising:
a mouth guard comprising pliable material configured to protect teeth and mouth tissue; and
at least one sensor integrated into said mouth guard, wherein said at least one sensor is selected to measure at least one blood constituent selected from the group consisting of blood sugar, blood oxygen, oxygen saturation level, blood acidity and carbon dioxide level.

2. The monitor of claim 1, further comprising a temperature sensor.

3. The monitor of claim 2, further comprising an alarm operatively connected to said at least one sensor or to said temperature sensor.

4. The monitor of claim 1, further comprising a pulse monitor.

5. The monitor of claim 1, further comprising means for measuring sodium or potassium level.

6. The device of claim 1, further comprising means for wirelessly transmitting sensor information to a computer for analysis.
